(19) United States
(12) Patent Application Publication
(10) Pub. No.: US 2022/0370546 A1
(43) Pub. Date: Nov. 24, 2022

(54) PEPTIDE, COMPOSITION, AND METHOD FOR TREATING, PREVENTING, OR AMELIORATING MOOD DISORDER

(71) Applicants: KYOTO UNIVERSITY, Kyoto (JP); Kazusa DNA Research Institute, Chiba (JP); KAMEDA SEIKA CO., LTD., Niigata (JP)

(72) Inventors: Kousaku OHINATA, Kyoto (JP); Shinto JO, Kyoto (JP); Hideyuki SUZUKI, Chiba (JP); Masaru SATO, Chiba (JP); Akira ITO, Niigata (JP); Yuki HIGUCHI, Niigata (JP)

(21) Appl. No.: 17/774,093

(22) PCT Filed: Nov. 5, 2020
(86) PCT No.: PCT/JP2020/041417

§ 371 Date: May 3, 2022

(30) Foreign Application Priority Data
Nov. 5, 2019 (JP) 2019-200911

(51) Int. Cl. A61K 38/08; A61K 38/06; A23L 33/18; C07K 7/06; A61P 25/24

(52) U.S. Cl. CPC A61K 38/08; A61K 38/06; A23L 33/18; C07K 7/06; A61P 25/24

(57) ABSTRACT

The present invention addresses the problem of providing a novel peptide that can be used to treat, prevent, or ameliorate mood disorders. The present invention provides a peptide that has an amino acid sequence set forth in SEQ ID NO: 1 or SEQ ID NO: 2 and is 3 to 5 (inclusive) amino acids long. The peptide may consist of an amino acid sequence set forth in SEQ ID NO: 1, SEQ ID NO: 2, SEQ ID NO: 3, or SEQ ID NO: 4.

### TECHNICAL FIELD

The present invention relates to a peptide, a composition, and a method for treating, preventing, or ameliorating mood disorders.

### BACKGROUND ART

An increase in mood disorders, such as diminished motivation and depression, is a problem that arises from the modern stress society. Anxiety is a cause of mood disorders, although it is inevitable and necessary as it alerts a living individual to avoid danger. An excessive degree of anxiety is involved in the onset of a mood disorder and the progression of the symptom, and development of foods and pharmaceuticals for the alleviation of anxiety has been expected.

For example, Patent Document 1 discloses that a certain dipeptide can be suitable for use as an anti-anxiety drug.
Patent Document 1: PCT International Publication No. WO2013/129220

### DISCLOSURE OF THE INVENTION

### Problems to be Solved by the Invention

However, there is a further need for functional materials that can be used to treat, prevent, or ameliorate mood disorders.

The present invention has been made in light of the circumstances, and an object of the present invention is to provide a novel peptide that can be used to treat, prevent, or ameliorate mood disorders.

### Means for Solving the Problems

The inventors have completed the present invention based on the findings that a peptide having a specific amino acid sequence provides a solution to the problem mentioned above. Specifically, the present invention provides the following aspects.

(1) A peptide comprising an amino acid sequence set forth in SEQ ID NO: 1 or 2 and being 3 or more and 5 or less amino acids long.

(2) The peptide according to aspect (1), consisting of an amino acid sequence set forth in SEQ ID NO: 1, 2, 3, or 4.

(3) A composition for use in treating, preventing, or ameliorating a mood disorder, the composition comprising the peptide according to aspect (1) or (2).

(4) The composition according to aspect (3), wherein the mood disorder is at least one selected from the group consisting of diminished motivation, depression, a depressive mood disorder, and symptoms based on any of the diminished motivation, the depression, and the depressive mood disorder.

(5) The composition according to aspect (3) or (4), being a pharmaceutical product.

(6) The composition according to aspect (3) or (4), being a food or beverage product.

(7) A method for treating, preventing, or ameliorating a mood disorder, the method comprising administrating the composition according to any one of aspects (3) to (5).

### Effects of the Invention

The present invention provides a novel peptide that can be used to treat, prevent, or ameliorate mood disorders.

### BRIEF DESCRIPTION OF THE DRAWINGS

FIG. 1 is a graph showing results of a tail suspension test using mice orally administered with different peptides.

### PREFERRED MODE FOR CARRYING OUT THE INVENTION

Hereinafter, embodiments of the present invention will be described in detail. It will be understood that the embodiments described below are not intended to limit the present invention.

### <Peptide of the Present Invention>

The peptide of the present invention comprises the amino acid sequence set forth in SEQ ID NO: 1 (QSQ) or the amino acid sequence set forth in SEQ ID NO: 2 (SQK) and is 3 or more and 5 or less amino acids long. It should be noted that hereinafter the amino acid sequences are shown from their N to C terminus with their N terminus on the left.

The inventors have conducted an investigation based on information resulting from simultaneous analysis of mixtures of a variety of peptides and information about the correlation between the structure and activity of known peptides that affect emotional behavior. As a result, the inventors have found new peptides that may be effective in treating mood disorders, which correspond to those defined above.

The peptide of the present invention may consist of the amino acid sequence set forth in SEQ ID NO: 1 or 2 (tripeptide) or have an amino acid sequence resulting from the addition of any amino acid or acids to the N and/or C terminus of the amino acid sequence set forth in SEQ ID NO: 1 or 2.

The amino acid sequence set forth in SEQ ID NO: 1 has a glutamine residue (Q) at the N terminus and also a glutamine residue (Q) at the C terminus. The amino acid sequence set forth in SEQ ID NO: 2 has a serine residue (S) at the N terminus and a lysine residue (K) at the C terminus.

The peptide of the present invention is up to 5 or less amino acids long, preferably up to 4 or less amino acids long. Most preferably, the peptide of the present invention is 3 amino acids long (namely, most preferably, the peptide of the present invention consists of the amino acid sequence set forth in SEQ ID NO: 1 or 2).

The peptide having an amino acid sequence resulting from the addition of an amino acid or acids to the N and/or C terminus of the amino acid sequence set forth in SEQ ID NO: 1 or 2 is preferably, but not limited to, a peptide consisting of the amino acid sequence set forth in SEQ ID NO: 3 (QSQSQ) or a peptide consisting of the amino acid sequence set forth in SEQ ID NO: 4 (SQSQK).

The amino acid sequence set forth in SEQ ID NO: 3 has two amino acid residues (QS) added to the N terminus of the amino acid sequence set forth in SEQ ID NO: 1 or two amino acid residues (SQ) added to the C terminus of the amino acid sequence set forth in SEQ ID NO: 1. The amino acid sequence set forth in SEQ ID NO: 4 has an amino acid residue (S) added to the N terminus and an amino acid residue (K) added to the C terminus of the amino acid sequence set forth in SEQ ID NO: 1 or has two amino acid residues (SQ) added to the N terminus of the amino acid sequence set forth in SEQ ID NO: 2.

As a result of the inventors' investigation, it has been suggested that the tripeptide (the peptide consisting of the amino acid sequence set forth in SEQ ID NO: 1 or 2), which forms the peptide of the present invention, should be a minimum unit for producing a motivation-enhancing effect. It has also been found that partial peptides 2 or less amino acids long (e.g., dipeptides such as QS, QK, and SQ) which constitute the peptide of the present invention cannot have a sufficient level of motivation-enhancing effect.

The peptide of the present invention can be obtained by chemical synthesis or hydrolysis of natural proteins or polypeptides.

The chemical synthesis method may be a known peptide synthesis method. Specifically, the chemical synthesis method may be a liquid- or solid-phase method commonly used for peptide synthesis. More specifically, the chemical synthesis method may be Fmoc method or Boc method. The synthesized peptides may be subjected to purification. The purification method may include, for example, ion-exchange chromatography, reverse-phase liquid chromatography, or affinity chromatography.

The hydrolysis method may include a method using a hydrolase or a method using a strong acid or a strong base.

The method using a hydrolase may be carried out using an animal-, plant-, or microorganism-derived hydrolase (e.g., trypsin, chymotrypsin, papain, pepsin, carboxypeptidase, thermolysin). An edible microorganism (e.g., baker's yeast, beer yeast, any other food yeast) may also be used as a hydrolase source.

As a non-limiting example, the hydrolysis using a hydrolase may be carried out at a suitable level of pH, adjusted depending on the enzyme used, at a temperature of about 30 to about 70° C. for a reaction time period of 30 minutes to 48 hours. The peptide of the present invention may be purified from the resulting liquid reaction product before use. When the hydrolyzed product is a food material, it may also be used as a food product without any modification or added to any other food material to form a food product.

The strong acid method may be carried out using, for example, hydrochloric acid, nitric acid, or sulfuric acid. The strong base method may be carried out using, for example, an alkali metal hydroxide (e.g., sodium hydroxide, potassium hydroxide, lithium hydroxide), an alkali metal carbonate (e.g., sodium carbonate, potassium carbonate), or an alkali metal hydrogen carbonate (e.g., sodium hydrogen carbonate, potassium hydrogen carbonate).

As a non-limiting example, the hydrolysis using a strong acid or base may be carried out in water in the presence of a strong acid or a strong base at a temperature of 1 to 100° C. for a reaction time period of 30 minutes to 48 hours. After being subjected to pH adjustment, the hydrolysis reaction product may be used without any modification or may be subjected to purification for isolation of the peptide of the present invention before use.

The amino acid sequence of the peptide resulting from various methods may be analyzed using a protein sequencer, which reads the amino acid sequence from the C terminus using Edman degradation, or analyzed by GC-MS or other methods.

### <Composition of the Present Invention>

The composition of the present invention includes at least the peptide of the present invention. The composition of the present invention may consist of the peptide of the present invention or may include any other component in addition to the peptide of the present invention.

Mood disorders can be treated, prevented, or ameliorated by taking the peptide of the present invention. Therefore, the composition of the present invention is advantageously used to treat, prevent, or ameliorate mood disorders.

As used herein, the term “mood disorder” means a mental disorder including a disorder of mood (or emotion). Specifically, the mood disorder may be at least one of diminished motivation, depression, a depressive mood disorder, and symptoms based on any of the above. Among the mood disorders, the present invention is particularly effective in treating, preventing, or ameliorating diminished motivation.

As used herein, the term “treat” means, for example, retarding the progression of mood disorders and curing the symptoms. The term “prevent” means, for example, inhibiting or retarding the onset of mood disorders. The term “ameliorate” means, for example, alleviating or reducing the symptoms of mood disorders.

The composition of the present invention may be prepared in any form. The composition of the present invention may be prepared in the form of a pharmaceutical product or a food or beverage product.

When the composition of the present invention is prepared in the form of a pharmaceutical product, it may be in the form of an orally or parenterally administrable agent. The composition of the present invention may be prepared, using the peptide of the present invention alone or in combination with a carrier, a diluent, or a vehicle, in the form of: tablets (e.g., uncoated tablets, sugar-coated tablets, foaming tablets, film-coated tablets, chewable tablets), capsules, troches, powders, fine granules, granules, liquids, suspensions, emulsions, pastes, creams, injections (including forms to be added to infusions, such as amino acid infusions or electrolyte infusions), enteric tablets, capsules, or sustained release preparations.

The carrier, diluent, or vehicle may be a material commonly used in the field of drug formulation and non-reactive with the peptide of the present invention. Examples of the carrier, diluent, or vehicle include lactose, glucose, mannitol, dextrin, cyclodextrin, starch, sucrose, magnesium metasilicate aluminate, synthetic aluminum silicate, sodium carboxymethyl cellulose, hydroxypropyl starch, calcium carboxymethyl cellulose, ion exchange resin, methylcellulose, gelatin, gum arabic, hydroxypropyl cellulose, hydroxypropyl methylcellulose, polyvinylpyrrolidone, polyvinyl alcohol, light anhydrous silicic acid, magnesium stearate, talc, tragacanth, bentonite, veegum, titanium oxide, sorbitan fatty acid esters, sodium lauryl sulfate, glycerin, glycerol esters of fatty acids, purified lanolin, glycerogelatin, polysorbate, macrogol, vegetable oil, wax, liquid paraffin, white petrolatum, fluorocarbon, nonionic surfactants, propylene glycol, and water.

When the composition of the present invention is prepared in the form of a food or beverage product, the food or beverage product may be in any form, such as beverages (e.g., coffee, cocoa, juice, soft drinks, mineral beverages, tea beverages, green tea, black tea, oolong tea, milk beverages, lactic acid bacteria beverages, yogurt drinks, carbonated drinks), rice snacks (e.g., rice crackers, rice biscuits, cubic rice crackers), gum, gummy candies, jelly, candies, cookies, crackers, biscuits, ices (e.g., ice cream, ice candies, sherbet, ice shavings), retort foods, or jelly foods (e.g., jelly, agar, jelly drinks).

The food or beverage product according to the present invention may also be prepared in the form of what is called a health food product, a functional food product, a dietary supplement, a supplement, a food for specified health use, a food with a functional claim, a food or combined food for medical uses (a type of food for special dietary use designated by Ministry of Health, Labor, and Welfare of Japan), or a food for the elderly (a type of food for special dietary use designated by Ministry of Health, Labor, and Welfare of Japan).

The content of the peptide of the present invention in the composition of the present invention may be appropriately adjusted depending on the desired effect. For example, the composition may preferably contain 0.01% by mass or more, more preferably 1.00% by mass or more of the peptide of the present invention. The composition may also contain preferably 100% by mass or less, more preferably 90% by mass or less of the peptide of the present invention. When the composition further contains peptides other than the peptide of the present invention, the value shown above should be calculated as the content of the peptide of the present invention based on the mass of the composition.

The content of any component other than the peptide of the present invention in the composition of the present invention may be appropriately adjusted depending on the type of the component, the form of the composition, and the desired effect.

The composition of the present invention may be administered by any method. The administration method may be oral administration or parenteral administration (e.g., injection). Preferably, the composition of the present invention is orally administered, so that the advantageous effect of the present invention can be easily produced.

The dose of the composition of the present invention varies depending on the administration method and the condition, age, and other characteristics of the subject. For example, the dose of the peptide of the present invention per adult per day is preferably 0.01 mg/kg to 500 mg/kg, more preferably 0.05 mg/kg to 100 mg/kg, even more preferably 0.1 mg/kg to 30 mg/kg. Within the range shown above, the higher the dose, the more likely the advantageous effect of the present invention tends to be produced.

The composition of the present invention may be produced by a known method, which may be selected depending on the desired form.

### <Method for Treating, Preventing, or Ameliorating Mood Disorders>

Mood disorders can be treated, prevented, or ameliorated by administering the composition of the present invention to the subject.

The administration method may be appropriately selected depending on the form of the composition.

The frequency of administration, the interval of administration, and the dose may be appropriately selected depending on the condition of the subject (e.g., symptom, age, body weight).

The subject may be, but not limited to, a human or a non-human mammal (e.g., dog, cat, a domestic animal (e.g., cattle, pig, sheep, goat)).

### Examples

Hereinafter, the present invention will be specifically described with reference to examples, which are not intended to limit the present invention.

### <Preparation of Peptides>

The eight peptides shown below were prepared by Fmoc synthesis and then reverse-phase HPLC purification. Peptides 1 to 4 each correspond to the peptide of the present invention.

Peptide 1: A peptide consisting of the amino acid sequence represented by SEQ ID NO: 1 (QSQ)
Peptide 2: A peptide consisting of the amino acid sequence represented by SEQ ID NO: 2 (SQK)
Peptide 3: A peptide consisting of the amino acid sequence represented by SEQ ID NO: 3 (QSQSQ)
Peptide 4: A peptide consisting of the amino acid sequence represented by SEQ ID NO: 4 (SQSQK)
Peptide 5: A peptide consisting of the amino acid sequence represented by SEQ ID NO: 5 (QS)
Peptide 6: A peptide consisting of the amino acid sequence represented by SEQ ID NO: 6 (QK)
Peptide 7: A peptide consisting of the amino acid sequence represented by SEQ ID NO: 7 (SQ)
Peptide 8: A peptide consisting of the amino acid sequence represented by SEQ ID NO: 8 (QSQSQK)

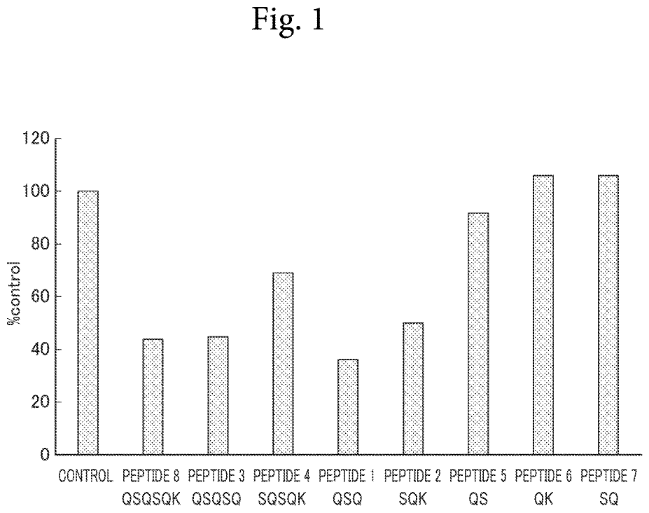

Each of the prepared peptides was administered to mice (ddY mice (5-week-old male mice 24 to 28 g in body weight)) by the method shown below. The mice were then subjected to a tail suspension test for the evaluation of a motivation-enhancing effect. The results are shown in FIG. 1.

### <Administration of Peptide>

Each peptide was dissolved in saline. The solution was orally administered to mice (n=5) such that each of peptides 3, 4, and 8 was administered in a dose of 0.3 mg/kg body weight and each of peptides 1, 2, 5, 6, and 7 was administered in a dose of 0.2 μmol/kg body weight. Control mice (n=5) were orally administered with only saline. Each of the mice was subjected to a tail suspension test in which the peptide of the present invention was evaluated for its efficacy to enhance motivation.

### <Tail Suspension Test: Evaluation of Efficacy to Enhance Motivation>

Thirty minutes after the administration of the peptide, each mouse was suspended with its tail fixed 30 cm above the floor. Subsequently, during a period of 6 minutes from the start of the test (0 minutes), the time (immobility time) during which the mouse remained in a motionless state, observed after the start of escape behavior, was measured, and the measurements were averaged. The motionless state is known as “despair state”. The shorter immobility time can be evaluated as indicating that the despair state is more alleviated and motivation is more enhanced. This means that materials effective in enhancing motivation in this test may be effective in treating, preventing, or ameliorating mood disorders.

### <Results>

The results shown in FIG. 1 are the values relative to the immobility time of the control group, which is normalized to 100%, with respect to the mice administered with each peptide. In FIG. 1, the smaller value can be evaluated as indicating that the despair state is more alleviated and motivation is more enhanced.

FIG. 1 shows that the mice administered with the peptide of the present invention (one of peptides 1 to 4) showed an immobility time shorter than that shown by the control group. Therefore, the peptide of the present invention has been demonstrated to be effective in enhancing motivation and to be useful for treating or preventing mood disorders.

Moreover, specific tripeptides (peptides 1 and 2) among the peptides according the present invention had a particularly high level of motivation-enhancing effect.

On the other hands, the mice administered with the dipeptides (peptides 5 to 7) having the amino acid sequences represented by QS, QK, and SQ, respectively, showed almost no decrease in immobility time as compared to the control group.

Those results suggest that the peptide of the present invention should have a motivation-enhancing effect and that the tripeptide (peptide 1 or 2), which forms the peptide of the present invention, should be a minimum unit for producing a motivation-enhancing effect.

1. A peptide comprising an amino acid sequence set forth in SEQ ID NO: 1 or 2 and being 3 or more and 5 or less amino acids long.

2. The peptide according to claim 1, consisting of an amino acid sequence set forth in SEQ ID NO: 1, 2, 3, or 4.

3. A composition for use in treating, preventing, or ameliorating a mood disorder, the composition comprising the peptide according to claim 1.

4. The composition according to claim 3, wherein the mood disorder is at least one selected from the group consisting of diminished motivation, depression, a depressive mood disorder, and symptoms based on any of the diminished motivation, the depression, and the depressive mood disorder.

5. The composition according to claim 3, being a pharmaceutical product.

6. The composition according to claim 3, being a food or beverage product.

7. A method for treating, preventing, or ameliorating a mood disorder, the method comprising administrating the composition according to claim 3.


SEQUENCE LISTING (8 sequences)

SEQ ID NO: 1 - protein, 3 aa, Artificial Sequence
  synthesized peptide
QSQ

SEQ ID NO: 2 - protein, 3 aa, Artificial Sequence
  synthesized peptide
SQK

SEQ ID NO: 3 - protein, 5 aa, Artificial Sequence
  synthesized peptide
QSQSQ

SEQ ID NO: 4 - protein, 5 aa, Artificial Sequence
  synthesized peptide
SQSQK

SEQ ID NO: 5 - protein, 2 aa, Artificial Sequence
  synthesized peptide
QS

SEQ ID NO: 6 - protein, 2 aa, Artificial Sequence
  synthesized peptide
QK

SEQ ID NO: 7 - protein, 2 aa, Artificial Sequence
  synthesized peptide
SQ

SEQ ID NO: 8 - protein, 6 aa, Artificial Sequence
  synthesized peptide
QSQSQK